(19) United States
(12) Patent Application Publication
(10) Pub. No.: US 2024/0296264 A1
(43) Pub. Date: Sep. 5, 2024

(54) DETERMINATION OF SURFACES AND VOLUMES WORTH PROTECTING IN ADDITIVE/SUBTRACTIVE MANUFACTURING JOBS WITH NEURAL NETWORKS

(71) Applicants: DENTSPLY SIRONA Inc., York, PA (US); SIRONA Dental Systems GmbH, Bensheim (DE)

(72) Inventor: Christian STAHL, Bensheim (DE)

(21) Appl. No.: 18/578,357

(22) PCT Filed: Jul. 12, 2022
(86) PCT No.: PCT/EP2022/069382

§ 371 Date: Jan. 11, 2024

(30) Foreign Application Priority Data
Jul. 12, 2021 (EP) 21184956.7

(51) Int. Cl. G06F 30/27; A61C 13/00; A61C 13/34; B33Y 50/00; B33Y 80/00

(52) U.S. Cl. CPC G06F 30/27; A61C 13/0004; A61C 13/34; B33Y 50/00; B33Y 80/00

(57) ABSTRACT

A computer-implemented method for the automatic generation of component-describing data for use in a preparation of additive/subtractive manufacturing jobs for dental components such as splints, denture bases, models, restorations such as bridges and crowns, in which for each at least one component type a specialized pre-trained neural network is used for setting surface and/or volume attributes of the dental component, in which the surface and volume attributes describe the accuracy and quality requirements of construction elements of the dental components with regard to the intended use, in which the accuracy and quality requirements comprise at least one of the following: geometric dimensional accuracy, mechanical strength, surface texture color, and the avoidance of the attachment of support elements, in which the neural network has been pre-trained by means of dental components for which the surface and/or volume attribution has already been carried out.

The entire content of the priority application EP21184956.7 is hereby incorporated by reference to this international application under the provisions of the PCT.

### TECHNICAL FIELD OF THE INVENTION

The present invention relates to additive/subtractive manufacturing processes of dental components.

### BACKGROUND OF THE INVENTION

Prior art CAD/CAM software for dental components uses surface attributes to offer high quality preparation of 3D printing or milling jobs. These surface attributes mark, for example, sensitive/functional surface regions on which no support elements are to be placed during 3D printing in order to avoid manual postprocessing on these regions. The surface attributes can be set automatically if suitable input data is available, e.g., a sufficiently granular subdivision of the component into construction elements that has already taken place in the construction step.

If the input data does not contain suitable information for automatic setting of the surface attributes in the CAD/CAM software, especially if the component data contains only geometry information, which is often the case, the surface attributes must be added subsequently. Typically, the user has to add these surface attributes manually. For this purpose, a “Painter tool” is provided in some CAD/CAM software, for example. However, the process of manual attribution using the “Painter tool” is time-consuming and potentially error-prone since the user defines the surface attributes at his own discretion and not necessarily according to designated or expected aspects.

### DISCLOSURE OF THE INVENTION

One objective of the present invention is to provide a computer-implemented method and CAD/CAM software for high-quality automatic preparation of additive/subtractive manufacturing jobs for dental components.

Another objective of the present invention is to provide a method and CAD/CAM software to set the surface and volume attributes of a dental component by neural networks.

These objectives are achieved by the method according to claim 1, and the CAD/CAM software according to claim 8. The subject-maters of the dependent claims relate to further developments as well as preferred embodiments.

The method according to the present invention and the corresponding CAD/CAM software are used for high-quality automatic preparation of additive/subtractive manufacturing jobs for dental components of various types, such as splints, denture bases, models, restorations such as bridges and crowns, among others. The CAD/CAM software either features neural networks for component type classification, or the type of components is already known from other sources. Further based on this, according to the invention, a specialized pre-trained neural network is used for the surface and/or volume attribution of the component for each at least one component type, i.e., several neural networks may be used for several component types. The surface and volume attributes describe sensitive/functional surfaces and volumes, in particular the accuracy and quality requirements of the various construction elements of the components, where construction elements each comprise one or more surface or volume regions of the component. Such sensitive/functional construction elements are, for example, the preparation margins on crowns and bridges or the drill spoon supports on drill templates and are generally specific to the component type.

The present invention specifically provides a computer-implemented method for the automatic generation of component-describing data for use in a preparation of additive/subtractive manufacturing jobs for dental components such as splints, denture bases, models, restorations such as bridges and crowns, wherein for each at least one component type a specialized pre-trained neural network is used for setting surface and/or volume attributes of the dental component, wherein the surface and volume attributes describe the accuracy and quality requirements of construction elements of the dental components with regard to the intended use, wherein the accuracy and quality requirements comprise at least one of the following: geometric dimensional accuracy, mechanical strength, surface texture color, and the avoidance of the attachment of support elements, and wherein the neural network has been pre-trained by means of dental components for which the surface and/or volume attribution has already been carried out.

The present invention also provides method of producing a dental component by an additive/subtractive manufacturing method using the component describing data generated as mentioned above.

Test and customer cases from a CAD/CAM software can serve as training data for the neural network, in which the surface and volume attributes are set with the CAD/CAM software based on construction elements known from the construction step of the components or set professionally in a manual way through expertise.

An advantageous effect of the invention is that through the method or the CAD/CAM software, the process of attributing surface and volume attributes can be performed automatically using neural networks according to the designated aspects of the component in an professional way. Such CAD/CAM software saves manual labor time and is less error-prone because of the use of pre-trained neural networks.

### BRIEF DESCRIPTION OF THE DRAWING

In the following description, the present invention will be explained in more detail by means of embodiments with reference to the drawing, whereby

FIG. 1—shows a component with some construction elements.

The reference numbers shown in the drawing designate the elements listed below, which are referred to in the following description of the exemplary embodiments.
1. Component
2,2′,2″ Construction element
3. Guide sleeve/bush

The method according to the present invention is explained in more detail below. The process according to the invention can be implemented through a CAD/CAM software.

The CAD/CAM software according to the invention allows high-quality automatic preparation of 3D printing or milling jobs for dental components such as splints, denture bases, models, restorations such as bridges and crowns. The CAD/CAM software knows the component type (e.g. splint, denture base, model, etc.) for each component, e.g. by using a neural network for component type classification or from another source. For each at least one component type, a specialized pre-trained neural network is further used for surface and/or volume attribution of the component. The surface and volume attributes describe the desired or necessary accuracy requirements and quality requirements of the construction elements of the components. Accuracy requirements and quality requirements include properties such as geometric dimensional accuracy, mechanical strength, surface finish/texture, color, and avoiding the attachment of support elements during 3D printing, etc.

The construction elements can also have characteristic properties within the variations of a component type, such as morphology, position within the component, environmental morphology, based on which they can be classified using the neural networks.

The construction elements are e.g., drill spoon supports on drill templates, bases/sockets on models, tooth pockets in denture bases, etc.

Test and customer cases from the CAD/CAM software can serve as training data, in which the surface and volume attributes are set by the CAD/CAM software based on construction elements known from the construction step of the components or set professionally in a manual way through expert knowledge.

The training datasets are used to train one or more, component type specific neural networks. The datasets can also be used for visualization on a display.

The CAD/CAM software is provided on a storage medium as program code and can be executed on a computer system. The CAD/CAM software can preferably also control the additive/subtractive manufacturing device e.g., a 3D printer or a milling machine. The computer system preferably comprises a user interface for the input of data describing the component geometry and/or training data relevant to the components.

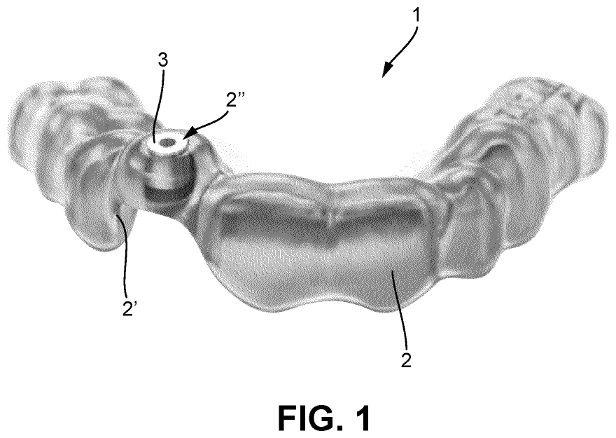

FIG. 1 shows a component (1), in particular a drilling template. The drilling template has an opening for receiving a guide sleeve (3) where a drill or an endodontic file can be guided. At the points where the guide sleeve (3) rests, the surface attribute, e.g., the accuracy requirement for manufacturing, can be set as high to enable precise placement of the guide sleeve (3). The different construction elements (2,2′,2′) of the drilling template comprise respectively, for example, the lower surface of the drilling template, the upper/outer surface of the drilling template and the inner surface of the opening in the drilling template which serves as a bearing/support surface for the guide sleeve (3). At the upper surface of the drilling template (surgical guide), in contrast to the lower surface where the drilling template rests on the tooth, the surface attribute e.g. the accuracy requirement can be set low. The pre-trained neural networks recognize the construction elements (2,2′,2″) for the component (1) and assign them the surface and/or volume attributes with corresponding accuracy requirements in accordance with the intended uses.

Instead of surface attributes or in addition to surface attributes, volume attributes can be used in a similar way whereby the additive/subtractive manufacturing (e.g. milling or 3D printing) of the volume regions is performed with the corresponding volume attributes describing the accuracy requirement.

In a further embodiment, the CAD/CAM software enables the region to be hollowed out to be marked by attributes in the case of models that have not been hollowed out and the model to be hollowed out on the basis of this marking. For this purpose, the pre-trained neural networks set corresponding surface and/or volume attributes of the area to be hollowed out.

Surface and/or volume attributes can take on values or characteristics that determine the accuracy and quality requirements for additive/subtractive manufacturing. To meet these requirements, the CAD/CAM software can then selectively make specific adjustments in the manufacturing process and/or preparation of the manufacturing process in these regions, e.g. variation of layer thickness, exposure dose, mask type or tool type; forcing presence/absence of support elements, etc.

1. Computer-implemented method for the automatic generation of component-describing data for use in a preparation of additive/subtractive manufacturing jobs for a dental component comprising:
setting, for each at least one component type, surface and/or volume attributes of the dental component, using a specialized pre-trained neural network,
wherein the surface and/or volume attributes describe the accuracy and quality requirements of construction elements of the dental components with regard to the intended use,
wherein the accuracy and/or quality requirements comprise at least one of the following: geometric dimensional accuracy, mechanical strength, surface texture color, and the avoidance of the attachment of support elements,
wherein the neural network has been pre-trained by means of other dental components for which the surface and/or volume attribution has already been carried out.

2. Computer-implemented method according to claim 1, wherein the construction elements have characteristic properties within the variations of a component type, selected from the list consisting of morphology, position within the dental component, and environmental morphology, on the basis of which they can be classified with the aid of the neural network and provided with corresponding attributes.

3. Computer-implemented method according to claim 1, wherein, the construction element to be attributed is at least one of the following: drill spoon support on a drill template, base/socket in models, tooth pocket in denture bases.

4. Computer-implemented method according to claim 1, wherein test and customer cases from a CAD/CAM software serve as training data, in which the surface and/or volume attributes are at least partially set manually and/or at least partially set with the CAD/CAM software on the basis of distinguishable construction elements.

5. Computer-implemented method according to claim 1, wherein a component type classification is performed using a neural network based on triangulation nodes and/or triangles of the dental components.

6. (canceled)

7. (canceled)

8. A non-transitory computer-readable storage medium storing a program, comprising instructions which when executed by a computer causes the computer to:
set, for each at least one component type, surface and/or volume attributes of the dental component, using a specialized pre-trained neural network,
wherein the surface and/or volume attributes describe the accuracy and quality requirements of construction elements of the dental component with regard to the intended use,
wherein the accuracy and/or quality requirements comprise at least one of the following: geometric dimensional accuracy, mechanical strength, surface texture color, and the avoidance of the attachment of support elements,
wherein the neural network has been pre-trained by means of other dental components for which the surface and/or volume attribution has already been carried out.

9. (canceled)
